(19) United States
(12) Patent Application Publication
(10) Pub. No.: US 2015/0157679 A1
(43) Pub. Date: Jun. 11, 2015

(54) HERBAL FORMULATION FOR TREATING A MENSTRUAL-RELATED CONDITION

(71) Applicant: PT. SOHO INDUSTRI PHARMASI, Jakarta (ID)

(72) Inventor: Alex Choon Hoi Liew, Ashford (AU)

(21) Appl. No.: 14/418,425

(22) PCT Filed: Jul. 30, 2013
(86) PCT No.: PCT/AU2013/000840

§ 371 Date: Jan. 29, 2015

Related U.S. Application Data
(60) Provisional application No. 61/677,147, filed on Jul. 30, 2012.

(51) Int. Cl. A61K 36/64; A61K 36/232; A61K 36/282; A61K 36/54; A61K 36/8905; A61K 36/65; A61K 36/708; A61K 36/484; A61K 36/284; A61K 36/236; A61K 36/076

(52) U.S. Cl. CPC A61K 36/64; A61K 36/236; A61K 36/232; A61K 36/282; A61K 36/54; A61K 36/076; A61K 36/65; A61K 36/708; A61K 36/484; A61K 36/284; A61K 36/8905; A61K 2236/33

(57) ABSTRACT

A formulation for treating a menstrual-related condition comprising a combination of the following herbs or extract(s) thereof: Radix rehmanniae praeparata, Rhizoma ligustici chuanxiong, Radix angelicae sinensis, Folium artemisiae argyi, Ramulus cinnamomi, Poria cocos and Radix paeoniae alba.

### PRIORITY DOCUMENTS

The present application claims priority from:

U.S. Provisional Patent Application No. 61/677,147 titled “A herbal formulation” and filed on 30 Jul. 2012 The content of this application is hereby incorporated by reference in its entirety.

### TECHNICAL FIELD

The present invention relates to a formulation for treating menstrual-related conditions in women.

### BACKGROUND

Menstrual-related pain and symptoms, including conditions such as endometriosis, dysmenorrhoea (painful periods), dyspareunia (pain during sexual intercourse), irregular menstrual bleeding including amenorrhoea (ie abnormally reduced and/or absent menstrual period) and/or menorrhagia (ie abnormally heavy and/or prolonged menstrual period), and/or pelvic or lower abdominal pain, are common in women of child-bearing age.

Endometriosis is a chronic disease with a poorly understood aetiology that is almost certainly multi-factorial. Diagnosis cannot be confirmed without performing surgery such as a laparoscopic procedure, but it is thought to affect at least 1 in 20 women of reproductive age. Many women suffer for years before a conclusive diagnosis is made and, following diagnosis, many continue to suffer irrespective of the nature of the treatment that they have received. Women with endometriosis usually present with intense menstrual-related pain and symptoms, menstrual-related conditions and/or infertility. Other symptoms of endometriosis may include abnormal menstrual bleeding (such as heavy bleeding, clotting, prolonged bleeding, irregular bleeding or premenstrual spotting); bowel disturbances such as dyschesia (pain with defaecation), diarrhoea, constipation and bleeding from the bowel (rectal bleeding); dysuria (painful urination) and haematuria (blood with urination); and/or pain in the lower back, thigh and/or groin.

No conventional medical or surgical treatments, apart from an artificial menopause, have been shown to prevent recurrences in endometriosis. Further, conventional treatments can be problematic as they often compromise fertility and are associated with side-effects such as accelerated bone loss, hot flushes, vaginal dryness, headaches, increased low-density lipoprotein cholesterol levels, weight gain, oily skin, mood swings, reduced libido, and/or increased thrombo-embolic risk. Consequently, many women seek alternative remedies for endometriosis and other menstrual-related conditions.

Chinese medicine provides remedies for menstrual-related disorders including menstrual-related pain and symptoms thought to be caused by an imbalance in ‘Yin-Yang’, and blockages in ‘Qi’. In Traditional Chinese medicine (TCM), menstrual-related conditions including endometriosis are attributed to a combination of kidney Yang or Yin vacuity and liver Qi stagnation. TCM teaches that the pathogenesis of endometriosis is related to ‘internal coldness’ with resulting ‘blocked liver Qi’, creating a predisposition to menstrual-related conditions with disturbed blood Qi in the lower jiao′ (lower abdomen) and in the female reproductive system. Another cause of endometriosis according to TCM may be kidney Qi deficiency with liver Qi stagnation, which affects the Chong and Ren channels (ie the main ‘meridians’ that transmit blood Qi to the lower reproductive system).

Chinese medicinal plant products are made from specifically selected whole plants or parts of plants, such as leaves, branches, stems, roots, tubers or bark, which are harvested from particular flowers, shrubs or trees, etc, which are termed ‘herbs’. Suitable herbs can be prescribed as specific formulations in accordance with TCM diagnosis of the pathological condition. The herbs may, for example, act to correct the imbalance of Yin-Yang in the body, nourish blood Qi circulation and improve blood or hormonal deficiencies. TCM treatment of menstrual-related conditions may promote blood Qi circulation, eliminate blood stasis and nourish the kidney and liver (St and Yang, 2000; Liu and. Wu, 1999).

Chinese herbs, when used within recommended dosages and indications are considered safe and cause few side-effects, even when used for a prolonged period of time (Hsu, 1986; Chi, 1994; Schwartz, 1994). However, increasing indiscriminate use of Chinese herbs in various countries has created concern, as inappropriately high dosages have been reported to cause side-effects and can interact with conventional drugs to cause adverse effects (Gottlieb, 2000; Tomlinson et al., 2000).

There has been a lack of rigorous clinical evidence regarding safety and benefits of Chinese medicine to treat menstrual-related conditions because of inadequate controlled study methodologies (Chi, 1994; He et al., 2006; Kotani et al., 1997), and there is no substantial clinical evidence that the TCM formulations are effective at treating menstrual-related conditions. The present inventor has found that clinical results using classical TCM formulations to treat menstrual-related conditions are inconsistent. Moreover, TCM dictates that the formulations should be varied in accordance with the oestrous cycle, which increases the difficulty to the subject in managing her own treatment. There are also reservations amongst women regarding the presentation of Chinese medications, which are often provided as a decoction or as rolled pills; and moreover, these traditional methods of preparation and decoction of TCM formulations do not provide formulations possessing consistent potency.

The present inventor has determined a herbal formulation that effectively treats endometriosis and other menstrual-related conditions, and conducted a stringent randomised clinical trial to evaluate this formulation of Chinese herbs to treat women with laparoscopy-proven endometriosis.

### SUMMARY

In a first aspect, the present invention provides a formulation comprising a combination of herbs or extract(s) thereof for treating a menstrual-related condition, the combination comprising Radix rehmanniae praeparata, Rhizoma ligustici chuanxiong, Radix angelicae sinensis, Folium artemisiae argyi, Ramulus cinnamomi, Poria cocas and Radix paeoniae alba; optionally in combination with a pharmaceutically-acceptable carrier or excipient.

In an embodiment, the combination further comprises at least one herb or extract thereof selected from the group consisting of Cortex moutan radicis, Radix paeoniae rubra, Rhizoma atractylodis macrocephalae, Radix et rhizoma rhei, Radix bupleuri, Rhizoma cyperi and Radix glycyrrhizae praeparata.

Preferably, the formulation comprises extract(s) derived from the combination of herbs. Preferably, the formulation comprises an extract derived from the combination of herbs, wherein the combination of herbs are mixed prior to extraction and extracted together. Preferably, the extract(s) is prepared by immersing the combination of dried herb(s) in an alcohol, macerating the immersed dried herb(s) to obtain a herbal alcohol solution, and then drying the herbal alcohol solution to obtain the extract(s).

In a second aspect, the present invention provides a method of treating a menstrual-related condition comprising administering to a female subject suffering from the menstrual-related condition an effective amount of the formulation of the first aspect.

In an embodiment, the method reduces pain associated with the menstrual-related condition. In an embodiment, the menstrual-related condition is selected from the group consisting of endometriosis, dysmenorrhoea, irregular menstrual bleeding, and dysparaeunia. Preferably, the formulation is administered throughout the oestrous cycle. Preferably, the formulation is administered orally.

### BRIEF DESCRIPTION OF FIGURES

FIG. 1 provides a copy of the Participant Survey (PS) Form I that each patient filled out by self assessment at baseline (ie immediately prior to commencing treatment), and at one, two, three, four, five and six months of treatment, and additionally at eight months (ie two months after treatment was completed), which required each patient to assess (Part A) their pain intensity (measured using a visual analogue scale (VAS)), and (Part B) restriction of activities (measured using the Likert scale) and safety of treatment (measured by monitoring for side effects);

FIG. 2 provides a participant flow chart detailing the number of patients that completed the trial; and

FIG. 3 provides a chart showing mean pain intensity during the trial, with the shaded area illustrating the two-month medication-free period.

### DETAILED DESCRIPTION

Various Chinese herbs and herbal formulations were initially trialled on women with menstrual-related conditions in accordance with Traditional Chinese Medicine (TCM); however, the benefits of the herbs were initially found to be inconsistent. The present inventor eventually determined a formulation comprising a combination of herbs selected on the basis of TCM that effectively treats menstrual-related conditions and/or symptoms, Advantageously, the formulation is considered to be safe and can optionally be administered throughout the oestrous cycle. The formulation comprises a combination of at least seven herbs, particularly, Radix rehmanniae praeparata, Rhizoma ligustici chuanxiong, Radix angelicae sinensis, Folium artemisiae argyi, Romulus cinnamotni, Poria cocos and Radix paeoniae alba.

Accordingly, in a first aspect, the present invention provides a formulation comprising a combination of herbs or extract(s) thereof for treating a menstrual-related condition, the combination comprising Radix rehmanniae praeparata, Rhizoma ligustici chuanxiong, Radix angelicae sinensis, Folium artemisiae argyi, Ramulus cinnamomi, Poria cocas and Radix paeoniae alba, optionally in combination with a pharmaceutically-acceptable carrier or excipient.

The term ‘herb’ as used herein is intended to refer to a particular plant and/or a particular component of a particular plant, as would be understood by those skilled in the art. For example, those skilled in the art will understand that the term ‘radix’ refers to the root of a plant; that the term ‘rhizoma’ refers to the rhizome of a plant; that the term ‘ramulus’ refers to the twigs (or small woody parts or stems) of a plant; that the term ‘folium’ refers to the leaves of a plant; and that the term ‘cortex’ refers to the bark of a plant. Further, those skilled in the art will understand that ‘Poria cocas’ refers to a fungus, and that the herb is prepared from sclerotium (eg a hard-surfaced body of fungal cells). Each of the herbs mentioned herein are known to those skilled in the art.

The term ‘extract’ as used herein is intended to refer to component(s) of the herb(s) that can be derived from the herb(s) using particular processes including steeping, macerating, decocting, and/or compressing, etc. Such process may optionally or additionally include evaporating, and/or drying, etc, as would be understood by those skilled in the art. Preferably, the extract is derived from the herb using a process detailed herein.

The present inventor has found that administration of the above formulation effectively reduces pain and symptoms associated with menstrual-related conditions such as endometriosis and dysmenorrhoea (ie uterine pain associated with menstruation), irregular menstrual bleeding including amenorrhoea (ie abnormally reduced and/or absent menstrual period), menorrhagia (ie abnormally heavy and/or prolonged menstrual period), and/or dysparaeunia (ie pain during sexual intercourse). Further, the present inventor has found that when certain further herb(s) or extract(s) thereof are included in the combination, they enhance the effects of the above combination, advantageously resulting in a further reduction in pain and symptoms associated with menstrual-related conditions. Accordingly, in an embodiment, the combination further comprises at least one herb or extract thereof selected from the group consisting of Cortex moutan radicis, Radix paeoniae rubra, Rhizoma atractylodis macrocephalae, Radix et rhizoma rhei, Radix bupleuri, Rhizoma cyperi and Radix glycyrrhizae praeparata.

The present inventor has conducted a clinical trial that demonstrated the combination of herbs or extract(s) thereof comprising Radix rehmanniae praeparata, Rhizoma ligustici chuanxiong, Radix angelicae sinensis, Folium artemistae argyi, Romulus cinnamomi, Poria cocos, Radix paeoniae alba, Cortex moutan radicis, Radix paeoniae rubra, Rhizoma atractylodis macrocephalae, Radix et rhizoma rhei and Radix glycyrrhizae praeparata, optionally in combination with a pharmaceutically-acceptable carrier or excipient, is particularly effective at treating menstrual-related conditions, for example, by reducing the pain and symptoms associated with menstrual-related conditions. Accordingly, in an embodiment, the combination of herbs or extract(s) thereof for treating a menstrual-related condition further comprises Cortex moutan radicis, Radix paeoniae rubra, Rhizoma atractylodis macrocephalae, Radix et rhizoma rhei and Radix glycyrrhizae praeparata.

Moreover, the present inventor has found that when the above combination of herbs also includes Radix bupleuri and Rhizoma cyperi, the formulation has even further effectiveness at treating menstrual-related conditions. Accordingly, in an embodiment, the combination of herbs or extract(s) thereof for treating a menstrual-related condition further comprises the combination further comprising Cortex mouton radicis, Radix paeoniae rubra, Rhizoma atractylodis macrocephalae, Radix et rhizoma rhei, Radix bupleuri, Rhizoma cyperi and Radix glycyrrhizae praeparata.

The formulation may comprise the herbs or extract(s) thereof in any concentration providing that the formulation effectively treats menstrual-related conditions (ie the formulation effectively reduces pain and symptoms associated with menstrual-related conditions). For example, the combination may comprise approximately 2% to 30% w/w Radix rehmanniae praeparata, approximately 0.1% to 25% w/w Rhizoma ligustici chuanxiong, approximately 0.1% to 25% w/w Radix angelicae sinensis, approximately 0.1% to 20% w/w Folium artemisiae argyi, approximately 0.1% to 25% w/w Ramulus cinnamomi, approximately 0.1% to 25% w/w Poria cocos, approximately 0.1% to 25% w/w Radix paeoniae alba, approximately 0% to 20% w/w Cortex moutan radicis, approximately 0% to 20% w/w Radix paeoniae rubra, approximately 0% to 20% w/w Rhizoma atractylodis macrocephalae, approximately 0% to 15% w/w Radix et rhizoma rhei, approximately 0% to 15% w/w Radix bupleuri, approximately 0% to 15% w/w Rhizoma cyperi and approximately 0% to 15% w/w Radix glycyrrhizae praeparata. In an embodiment, the combination comprises approximately 7% to 26% w/w Radix rehmanniae praeparata, approximately 3% to 21% w/w Rhizoma ligustici chuanxiong, approximately 3% to 21% w/w Radix angelicae sinensis, approximately 0.1% to 12% w/w Folium artemisiae argyi, approximately 4% to 21% w/w Ramulus cinnamomi, approximately 4% to 21% w/w Poria cocos, approximately 3% to 19% w/w Radix paeoniae alba, approximately 0% to 13% w/w Cortex moutan radicis, approximately 0% to 13% w/w Radix paeoniae rubra, approximately 0% to 15% w/w Rhizoma atractylodis macrocephalae, approximately 0% to 9% w/w Radix et rhizoma rhei, approximately 0% to 9% w/w Radix bupleuri, approximately 0% to 9% w/w Rhizoma cyperi, and approximately 0% to 11% w/w Radix glycyrrhizae praeparata.

The formulation comprises at least seven of the above herbs in combination. For example, the combination may comprise 8% to 31% w/w Radix rehmanniae praeparata, 3% to 26% w/w Rhizoma ligustici chuanxiong, 3% to 26% w/w Radix angelicae sinensis, 0.1% to 17% w/w Folium artemisiae argyi, 5% to 26% w/w Ramulus cinnamomi, 5% to 26% w/w Poria cocos and 2% to 14% w/w Radix paeoniae alba. In an embodiment, the combination comprises 15% to 24% w/w Radix rehmanniae praeparata, 8% to 21% w/w Rhizoma ligustici chuanxiong, 8% to 21% w/w Radix angelicae sinensis, 1% to 12% w/w Folium artemisiae, 10% to 21% w/w Ramulus cinnamomi, 10% to 21% w/w Poria cocos and 7% to 19% w/w Radix paeoniae alba. Preferably, the combination comprises approximately 18% to 21% w/w Radix rehmanniae praeparata, approximately 13% to 16% w/w Rhizoma ligustici chuanxiong, approximately 13% to 16% w/w Radix angelicae sinensis, approximately 6%© to 7% w/w Folium artemisiae argyi, approximately 15% to 16% w/w Ramulus cinnamomi, approximately 15% to 16% w/w Poria cocos and approximately 12% to 14% w/w Radix paeoniae alba.

The combination of the above seven herbs may further comprise at least one herb or extract thereof selected from the group consisting of Cortex moutan radicis, Radix paeoniae rubra, Rhizoma atractylodis macrocephalae, Radix et rhizoma rhei, Radix bupleuri, Rhizoma cyperi and Radix glycyrrhizae praeparata.

In an embodiment, the formulation comprises twelve of the above herbs in combination. For example, the combination may comprise 2% to 22% w/w Radix rehmanniae praeparata, 0.1% to 20% w/w Rhizoma ligustici chuanxiong, 0.1% to 20% w/w Radix angelicae sinensis, 0.1% to 14% w/w Folium artemisiae argyi, 0.1% to 20% w/w Ramulus cinnamomi, 0.1% to 20% w/w Poria cocas, 0.1% to 18% w/w Radix paeoniae alba, 0.1% to 18% w/w Cortex moutan radicis, 0.1% to 18% w/w Radix paeoniae rubra, 0.1% to 20% w/w Rhizoma atractylodis macrocephalae, 0.1% to 14% w/w Radix et rhizoma rhei and 0.1% to 16% w/w Radix glycyrrhizae praeparata. In an embodiment, the combination comprises 7% to 17% w/w Radix rehmanniae praeparata, 5% to 15% w/w Rhizoma ligustici chuanxiong, 5% to 15% w/w Radix angelicae sinensis, 0.1% to 9% w/w Folium artemisiae argyi, 5% to 15% w/w Ramulus cinnamomi, 5% to 15% w/w Poria cocas, 3% to 13% w/w Radix paeoniae alba, 3% to 13% w/w Cortex mouton radicis, 3% to 13% w/w Radix paeoniae rubra, 5% to 15% w/w Rhizoma atractylodis macrocephalae, 0.1% to 9% w/w Radix et rhizoma rhei and 1% to 11% w/w Radix glycyrrhizae praeparata. Preferably, the combination comprises approximately 12% w/w Radix rehmanniae praeparata, approximately 10% w/w Rhizoma ligustici chuanxiong, approximately 10% w/w Radix angelicae sinensis, approximately 4% w/w Folium artemisiae argyi, approximately 10% w/w Ramulus cinnamomi, approximately 10% w/w Poria cocas, approximately 8% w/w Radix paeoniae alba, approximately 8% w/w Cortex moutan radicis, approximately 8% w/w Radix paeoniae rubra, approximately 10% w/w Rhizoma atractylodis macrocephalae, approximately 4% w/w Radix et rhizoma rhei and approximately 6% w/w Radix glycyrrhizae praeparata.

In an embodiment, the formulation comprises each of the above fourteen herbs. For example, the formulation may comprise 2% to 22% w/w Radix rehmanniae praeparata, 0.1% to 18% w/w Rhizoma ligustici chuanxiong, 0.1% to 18% w/w Radix angelicae sinensis, 0.1% to 14% w/w Folium artemisiae argyi, 0.1% to 19% w/w Ramulus cinnamomi, 0.1% to 19% w/w Poria cocos, 0.1% to 18% w/w Radix paeoniae alba, 0.1% to 18% w/w Cortex moutan radicis, 0.1% to 18% w/w Radix paeoniae rubra, 0.1% to 18% w/w Rhizoma atractylodis macrocephalae, 0.1% to 14% w/w Radix et rhizoma rhei, 0.1% to 14% w/w Radix bupleuri, 0.1% to 14% w/w Rhizoma cyperi and 0.1% to 16% w/w Radix glycyrrhizae praeparata. In another example, the formulation comprises 7% to 17% w/w Radix rehmanniae praeparata, 3% to 13% w/w Rhizoma ligustici chuanxiong, 3% to 13% w/w Radix angelicae sinensis, 0.1 to 9% w/w Folium artemisiae argyi, 4% to 14% w/w Ramulus cinnamomi, 4% to 14% w/w Poria cocos, 4% to 14% w/w Radix paeoniae alba, 3% to 13% w/w Cortex moutan radicis, 3% to 13% w/w Radix paeoniae rubra, 3% to 13% w/w Rhizoma atractylodis macrocephalae, 0.1% to 9% w/w Radix et rhizoma rhei, 0.1% to 9% w/w Radix bupleuri, 0.1% to 9% w/w Rhizoma cyperi and 1% to 11% w/w Radix glycyrrhizae praeparata. Preferably, the combination comprises approximately 12% w/w Radix rehmanniae praeparata, approximately 8% w/w Rhizoma ligustici chuanxiong, approximately 8% w/w Radix angelicae sinensis, approximately 4% w/w Folium artemisiae argyi, approximately 9% w/w Ramulus cinnamomi, approximately 9% w/w Poria cocas, approximately 8% w/w Radix paeoniae alba, approximately 8% w/w Cortex moutan radicis, approximately 8% w/w Radix paeoniae rubra, approximately 8% w/w Rhizoma atractylodis macrocephalae, approximately 4% w/w Radix et rhizoma rhei, approximately 4% w/w Radix bupleuri, approximately 4% w/w Rhizoma cyperi and approximately 6% w/w Radix glycyrrhfrae praeparata.

However, other concentrations of some or all of the herbs or extract(s) thereof may be suitable. Accordingly, those skilled in the art will understand that these ranges may be varied, providing that the formulation effectively treats menstrual-related conditions.

In any of the embodiments of the first aspect of the present invention, the formulation may consist solely of the combination of herbs or extract(s) thereof recited in that embodiment, optionally in combination with a pharmaceutically-acceptable carrier or excipient. In any of the embodiments of the first aspect of the present invention, the formulation may comprise active components that consist solely of the combination of herbs or extract(s) thereof recited in that embodiment (although the formulation may also include non-active components such as pharmaceutically acceptable filler, carrier, diluent and/or excipient, etc), rather than comprise the combination of herbs or extract(s) thereof recited in that embodiment.

In an embodiment, the formulation comprises extract(s) derived from the combination of herbs.

Extract(s) of the combination of herbs may be prepared using any method known to those skilled in the art, such as steeping, decocting, etc, providing the extract results in a formulation that effectively treats menstrual-related conditions. In an embodiment, the extract(s) may be prepared from dried herb(s) that have been immersed in a solvent or solution such that at least some of the active component(s) of the herb(s) are released into the solvent or solution (ie are ‘extracted’ from the herbs themselves). In an embodiment, the extract is prepared by immersing dried herb(s) in alcohol to form a herb/alcohol mixture, and then macerating the immersed herb(s) to obtain a herbal alcohol solution, and then vacuum drying the herbal alcohol solution to obtain the extract. Preferably, the extract contains at least some of the active ingredients of the herb(s) in a concentrated form. Small pieces of dried herb may be used to prepare the extract(s), for example, 1 to 4 cm in length, preferably 2 to 3 cm in length. Alternatively, the herbs may be ground. The alcohol may be any suitable alcohol known to those skilled in the art, for example, ethanol. The herb(s) may be immersed in the alcohol under any suitable conditions that facilitate the release of component(s) of the herb(s) into the alcohol. For example, the immersion may occur at a range of temperatures, under vacuum, or under vacuum for a period followed by a period at atmospheric pressure. For example, the extraction method may involve immersion of the herb(s) in 75% ethanol for to 60 minutes (eg 45 minutes) at 50° C. under vacuum conditions, and 3 to 4 hours at 50° C. without vacuum. The herb/alcohol mixture can optionally be macerated for a suitable period of time to enhance release of the active components of the herb(s) into the alcohol. Maceration can be performed using any suitable method known to those skilled in the art. For example, the herb/alcohol mixture may be placed in a tank, optionally containing earthenware, for a sufficient period of time, eg 24 to 72 hours. The resulting herbal alcohol solution can optionally be filtered. The herbal alcohol solution can then be concentrated (eg by drying or evaporation, etc), to obtain a concentrated herbal extract. The drying may be facilitated using, for example, a vacuum drier. In some embodiments, the herbal extract may be granulated to powder form.

In another example, the extraction method may involve immersion of herb(s) in 75% ethanol, optionally containing earthenware, for 3 hours at 50° C. under vacuum conditions, and then macerating the herb/alcohol mixture for a sufficient period of time, eg 5 to 7 days. The resulting herbal alcohol solution can optionally be pressed and/or filtered. The herbal alcohol solution can then be concentrated by evaporation and/or dried.

In some embodiments, the herbal extract may be milled to a desired particle size.

Those skilled in the art will appreciate that various changes to the extraction method may be made, providing the resulting extract effectively treats menstrual-related conditions.

Those skilled in the art will understand that various groups of herbs can be extracted together and/or herbs can be extracted individually, and such extracts can be mixed to form extract(s) of the combination of herbs. For example, a single extract may be extracted from all of the combination of herbs in a single extraction process, ie where all of the herbs of the combination are mixed prior to the extraction process and extracted together, resulting in a single extract. Alternatively, extracts may be derived from each herb individually, and the extracts can then be mixed together. In another alternative embodiment, a group of some of the herbs may be mixed together prior to extraction, and the resulting extract may be mixed with extract(s) of the other herbs of the combination, wherein the other herbs may be extracted individually and/or other herbs may be mixed to form another group of herbs that are extracted together (and combinations thereof); providing that extracts of all of the herbs of the combination are present in the formulation. Preferably, the formulation comprises an extract derived from the combination of herbs, wherein the combination of herbs are mixed prior to extraction and extracted together.

In an embodiment, the formulation may be combined with a pharmaceutically acceptable filler, carrier, diluent and/or excipient. The filler, carrier, diluent or excipient may be any suitable substance known to those skilled in the art, for example, dicalcium phosphate dibasic (DCPD); dibasic calcium phosphate, magnesium stearate, starches, sugars, lactose, sucrose, glucose, calcium carbonate, cellulose, cellulose derivatives or modified cellulose such as microcrystalline cellulose, hydroxypropyl cellulose, methyl cellulose, water, alcohol, gelatin, sodium starch glycolate, and/or fumed silica absorbent. Preferably, the filler, carrier, diluent or excipient may be magnesium stearate, DCPD, sodium starch glycolate, fumed silica absorbent, or a combination thereof.

The formulation may be provided in any form as long as the formulation is suitable for effectively treating menstrual-related conditions. For example, the formulation may be provided as a tablet, capsule, sachet, caplet, granules, powder, solution or elixir. Preferably, the formulation is provided in capsule or tablet form, or is within a sachet. Preferably, the formulation is suitable for oral administration. In an embodiment, the formulation may be provided without filler, carrier, diluent and/or excipient. For example, when the formulation is provided within a sachet, it may be provided without filler, carrier, diluent and/or excipient. Those skilled in the art will appreciate that other forms of the formulation may be provided without filler, carrier, diluent and/or excipient in some circumstances.

The present inventor carried out trials in the treatment of women with menstrual-related conditions employing a variety of herbs or extracts thereof, and combinations of herbs or extracts thereof, in accordance with Traditional Chinese Medicine (TCM). However, many herbs, and/or combinations thereof, were found to be unsuitable for the treatment of menstrual-related conditions, for example, due to inconsistent clinical results. However, the trials indicated that a formulation comprising a combination of herbs or extract(s) thereof, the combination comprising at least Radix rehmanniae praeparata, Rhizoma ligustici chuanxiong, Radix angelicae sinensis, Folium artemisiae argyi, Ramulus cinnamomi, Poria cocos and Radix paeoniae alba, would advantageously treat a menstrual-related condition. Moreover, the trials indicated that above combination further comprising at least one herb or extract thereof selected from the group consisting of Cortex moutan radicis, Radix paeoniae rubra, Rhizoma atractylodis macrocephalae, Radix et rhizoma rhei and Radix glycyrrhizae praeparata further enhanced treatment. The trials indicated that a formulation comprising Radix rehmanniae praeparata, Rhizoma ligustici chuanxiong, Radix angelicae sinensis, Folium artemisiae argyi, Ramulus cinnamomi, Poria cocos, Radix paeoniae alba, Cortex moutan radicis, Radix paeoniae rubra, Rhizoma atractylodis macrocephalae, Radix et rhizoma rhei and Radix glycyrrhizae praeparata was particularly effective at treating menstrual-related conditions. Additionally, the trials indicated that the inclusion of Radix bupleuri and Rhizoma cyperi in the formulation (that is, such that the formulation contained Radix rehmanniae praeparata, Rhizoma ligustici chuanxiong, Radix angelicae sinensis, Folium artemisiae argyi, Ramulus cinnamomi, Poria cocos, Radix paeoniae alba, Cortex moutan radicis, Radix paeoniae rubra, Rhizoma atractylodis macrocephalae, Radix et rhizoma rhei, Radix bupleuri, Rhizoma cyperi and Radix glycyrrhizae prcteparata) further unproved the efficiency of treating menstrual-related conditions.

The present inventor conducted a stringent, multi-centre, placebo-controlled randomised clinical trial to evaluate a formulation of the present invention comprising an extract derived from the combination of herbs Radix rehmanniae praeparata, Rhizoma ligustici chuanxiong, Radix angelicae sinensis, Folium artemisiae argyi, Ramulus cinnamomi, Poria cocos, Radix paeoniae alba, Cortex moutan radicis, Radix paeoniae rubra, Rhizoma atractylodis macrocephalae, Radix et rhizoma rhei and Radix glycyrrhizae praeparata to treat women with laparoscopy-proven endometriosis using suitable outcome measures and stringent inclusion-exclusion criteria. The trial showed that the formulation effectively reduced menstrual-related pain intensity and resulted in lessened restriction of activities in subjects. Additionally, the formulation was considered safe as there was no significant occurrence of drug sensitivity or reactions. Advantageously, the formulation was effective and safe when administered throughout the oestrous cycle for a prolonged period of time. Further, the formulation did not compromise natural fertility. Moreover, the effect of the treatment was sustained for a period of at least two months after completion of the treatment. The robustness of the trial indicates that the formulation is suitable for treating a range of benign menstrual-related conditions such as endometriosis, dysmenorrhoea, irregular menstrual bleeding including amenorrhoea and/or menorrhagia, and/or dysparaeunia.

Accordingly, in a second aspect, the present invention provides a method of treating a menstrual-related condition comprising administering to a female subject suffering from a menstrual-related condition an effective amount of the formulation of the first aspect.

The term ‘effective amount’ is intended to refer to any amount of the active components of the formulation that will elicit a beneficial or therapeutic effect in the subject (ie effectively reduce pain and symptoms associated with the menstrual-related condition). As used herein, the term ‘active components’ is intended to refer to the portion of the formulation that consists of the combination of herbs or extract(s) thereof, that is, excluding any filler, carrier, diluent and/or excipient, etc. For example, the effective amount of the formulation may be within a range between 95 mg and 11650 mg of the active components per subject per day. In an embodiment, the formulation is administered at a dosage within the range of 1850 mg to 7050 mg of the active components per subject per day. Preferably, the formulation is administered at a dosage within the range of 3150 mg to 5050 mg of the active components per subject per day. The dosage range of 3150 mg to 5050 mg of the active components may be particularly suitable for non-obese subjects, whereas a higher dosage, for example, of approximately 6300 mg of the active components may be more suitable for obese subjects. However, those skilled in the art will understand that the effective amount may vary from subject to subject, depending upon the subject's body weight, severity of symptoms, and sensitivity to the treatment, etc.

The formulation may be administered in various forms as described herein. The formulation may be in the form of, for example, a capsule, or tablet, or within a sachet, etc, which may be of various suitable sizes. For example, the capsules, tablets or sachets may conveniently be approximately 500 mg or 650 mg in weight, or other convenient sizes.

Where the combination of herbs are extracted as described herein and mixed with filler and excipients (etc) in a standard ratio to form the formulation, then the effective amount of the formulation can be calculated. For example, if a ratio of 3% filler, carrier, diluent and/or excipient, etc, is used in the formulation, then the effective amount of the formulation may be within a range between 100 mg and 12000 mg per subject per day. In an embodiment, the formulation is administered at a dosage within the range of 1950 mg to 7250 mg per subject per day. Preferably, the formulation is administered at a dosage within the range of 3250 mg to 5200 mg per subject per day. The dosage range of 3250 mg to 5200 mg may be particularly suitable for non-obese subjects, whereas a higher dosage, for example, of approximately 6500 mg may be more suitable for obese subjects.

In an embodiment, the formulation may, be administered as a particular number of standard portions of the formulation per day. For example, if the formulation is administered as 650 mg capsules containing a known ratio of filler and excipients (eg 3% filler, excipients, etc.), the formulation may be administered as three 650 mg capsules per subject per day, five 650 mg capsules per subject per day, or as six 650 mg capsules per subject per day, or as ten 650 mg capsules per subject per day, etc, to achieve the desired daily dosage of the formulation. Those skilled in the art will appreciate that other administration formats or dosages may be suitable (eg consisting of a particular number of 500 mg capsules, or alternatively tablets or sachets of a convenient weight, of the formulation per day, to achieve the desired daily dosage of the formulation).

The formulation can be administered in single or multiple doses. The entire dosage of the formulation may be administered once per day, or smaller doses of the formulation may be administered a number of different times per day, providing that the entire dosage is administered per day. For example, for a subject administered ten 500 mg capsules per day, the subject may be administered the ten capsules once per day; or five capsules twice per day; or 3 capsules twice per day plus four capsules once per day; etc. Those skilled in the art will appreciate that the dosage can be administered in other manners.

Preferably, the formulation is administered orally.

In an embodiment, the method reduces the symptoms associated with the menstrual-related condition. In an embodiment, the method reduces pain associated with the menstrual-related condition. Alternatively or additionally, the method increases the activity of the subject, that is, reduces restriction of the activity of the subject.

In an embodiment, the menstrual-related condition is selected from the group consisting of endometriosis, dysmenorrhoea, irregular menstrual bleeding including amenorrhoea or menorrhagia, and dyspareunia. In an embodiment, the formulation is administered throughout the oestrous cycle.

In a third aspect, the present invention provides a use of the formulation of the first aspect of the present invention for the treatment of a menstrual-related condition. In an embodiment, the use of the formulation is a use of an effective amount of the formulation for the treatment of a menstrual-related condition. In an embodiment, the formulation is in combination with a pharmaceutically-acceptable carrier or excipient. In an embodiment, the use comprises administering to a female subject suffering from the menstrual-related condition an effective amount of the formulation of the first aspect. In an embodiment, the formulation is administered at a dosage within the range of 1850 mg to 7050 mg of the active components per subject per day. In an embodiment, the formulation is administered at a dosage within the range of 3150 mg to 5050 mg of active components per subject per day. In an embodiment, the use reduces pain associated with the menstrual-related condition. In an embodiment, the menstrual-related condition is selected from the group consisting of endometriosis, dysmenorrhoea, irregular menstrual bleeding, and dyspareunia. In an embodiment, the formulation is administered throughout the oestrous cycle. In an embodiment, the formulation is administered orally.

In a fourth aspect, the present invention provides a use of the formulation of the first aspect of the invention in the manufacture of a medicament for the treatment of a menstrual-related condition. In an embodiment, the formulation is in combination with a pharmaceutically-acceptable carrier or excipient. In an embodiment, the manufactured medicament facilitates administration of an effective amount of the formulation for the treatment of a menstrual-related condition. In an embodiment, the manufactured medicament facilitates administration of the formulation at a dosage within the range of 1850 mg to 7050 mg of the active components per subject per day. In an embodiment, the manufactured medicament facilitates administration of the formulation at a dosage within the range of 3150 mg to 5050 mg of the active components per subject per day. In an embodiment, the menstrual-related condition is selected from the group consisting of endometriosis, dysmenorrhoea, irregular menstrual bleeding, and dyspareunia.

The present invention also provides a method for preparing the formulation of the first aspect of the invention as described herein.

Accordingly, in a fifth aspect, the present invention provides a method for preparing the herb extract(s) of the formulation of the present invention, wherein the method comprises the following steps: immersing a herb(s) in alcohol to obtain a herb/alcohol mixture, macerating the herb/alcohol mixture to obtain a herbal alcohol solution, and concentrating the herbal alcohol solution to produce the herb extract(s) of the formulation.

In an embodiment, the dried herbs or a combination thereof comprise all of the herbs of the formulation. In an embodiment, step b) of the method comprises macerating the herb/alcohol mixture in a tank containing earthenware. In an embodiment, the method further comprises filtering the herbal alcohol solution prior to step c) of the method. In an embodiment, step a) of the method comprises: immersing all of the combination of herbs of the first aspect of the invention in alcohol to obtain a herb/alcohol mixture. In an embodiment, step c) of the method comprises drying the herbal alcohol solution. In an embodiment, the formulation is in the form of a capsule, or tablet, or is within a sachet.

In accordance with Traditional Chinese Medicine and the Theory of Five Elements, the Earth (Element) is believed to be associated with the digestive system of humans and animals. Specifically, it has long been believed that decocting herbs in earthenware (pottery) containers permits the production of effective and soothing remedies. In an embodiment of the present invention, the herbs are extracted in tanks, for example, metallic tanks, in which earthenware pieces are added. While not wanting to be bound by theory, it is thought that the earthenware provides a catalytic action during the manufacturing process, enhancing efficient extraction and mixing of the active components, which may enhance their digestion and absorption.

The invention is hereinafter described by reference to the following non-limiting examples and accompanying figures.

### Examples

### Example 1

### Selecting combination of herbs for formulation

### Introduction

The main principle of TCM herbal pharmacopoeia is to treat the underlying cause of the condition (eg correcting Yin-Yang balance, blood Qi circulation, blocked liver Qi, internal coldness, etc) in accordance with Chinese medicine teachings, rather than to treat the symptoms (eg menstrual-related pain). In the initial stages of development of a herbal formulation for treating menstrual disorders, the present inventor found that classical Traditional Chinese Medicine (TCM) formulations provided clinical results that were inconsistent. Further, the present inventor considered that existing TCM practices did not sufficiently address underlying deficiencies in the women, such as intrinsic anaemia and exhaustion. Additionally, current thoughts in TCM dictate that formulations should be varied in accordance with the oestrous cycle, which is difficult for subjects to manage on their own, and moreover, there is no substantial clinical evidence that the TCM formulations are effective. Finally, the traditional methods of preparing and decocting TCM formulations do not provide consistent potency.

### Materials and Methods

A number of TCM herbs were considered and tested in combination, with the most efficacious combination of herbs incorporated into the final formulation to provide an advantageous action, for example, using herbs that should correct Yin-Yang balance while enhancing blood Qi circulation.

### Results and Discussion

In developing the formulation of the present invention, the objectives were: i) to provide an effective and safe herbal composition applicable throughout the oestrous cycle; and ii) to address the underlying causation of menstrual-related conditions and treat concomitant deficiencies in the women.

The actions associated With herbs selected for the formulation are as follows:

(1) The principal herb Radix rehmanniae praeparata (Shu di) acts together with Radix angelicae sinensis (Dang gui) to provide ‘Yin’ to improve the quality of blood and help correct the intrinsic anaemia, while assisting removal of ‘blood stasis’ (Fu, 1995; Page and Lawrence, 1999).
(2) Radix angelicae sinensis (Dang gui) additionally promotes blood circulation and regulates menstruation to alleviate pain due to ‘cold or blood deficiency’ (Ou, 1992; Hsu, 1986; Bensky and Gamble, 1986).
(3) Folium artemisiae argyi (Ai ye) helps to stop bleeding by ‘warming the meridian’ and ‘expelling cold to alleviate pain’ (Ou, 1992; Hsu, 1986; Fu, 1995), which is believed to enrich blood and address bleeding, as can occur in intra-pelvic endometriosis or lesions, and in heavy menstrual bleeding, particularly in exhausted or internally weak bodily constitutions (Ou, 1992; Hsu, 1986).
(4) Rhizoma ligustici chuanxiong (Chuan xiong) acts with Radix angelicae sinensis, Radix rehmanniae praeparata and Radix paeoniae alba (Bai shao) to supplement blood and improve intrinsic anaemia.
(5) Ramulus cinnamomi (Gui zhi) and Poria cocos (Fu ling), assisted by Cortex moutan radicis (Mu dan pi) and Radix paeoniae rubra (Chi shao), act to ‘warm the lower abdomen (lower jiao) and improve blood-Qi in the reproductive system and surrounding organs’ (Mu, 2000; Jiang et al., 1998).
(6) Radix paeoniae alba, together with Cortex moutan radicis, Radix paeoniae rubra and Radix et rhizoma rhei (Da huang), helps remove pathological blood stasis and toxic heat (Ou, 1992; Hsu, 1986; Shi et al., 2000; He et al., 1994).
(7) Rhizoma atractylodis macrocephalae (Bai zhu) acts on the spleen to remove accumulation of fluids and supplement vital energy, Qi (Ou, 1992; Hsu, 1986; Fu, 1995). When combined with Radix paeoniae alba and Radix angelicae sinensis, Rhizoma atractylodis macrocephalae may help prevent miscarriage by soothing the foetus in threatened abortion.
(8) Radix bupkuri (Chai hu) helps disperse the stagnant Liver Qi energy to enhance harmonious energy within the abdomen, such as removing stagnant Qi in the lower abdomen in dysmenorrhoea and menstrual disorders. When combined with Radix paeoniae alba, Radix angelicae sinensis and Rhizoma cyperi, Radix Bupleuri relieves associated abdominal and hypochondriac fullness and pain, as in liver Qi stagnation with cold extremities. It may relieve emotional disturbances such as irritability and emotional distress, and associated chest fullness and discomfort.
(9) Rhizoma cyperi (Xiang fu) disperses stagnated liver energy and enhances Qi-flow in the abdomen to alleviate pain in dysmenorrhoea and irregular menstruation due to stagnant Qi-blood flow within the lower abdomen, and to help prevent miscarriage.

When Chinese herbs are used in appropriate combinations, some herbs in the combination can enhance the effects of other herb(s) and/or nullify some of the intrinsic side-effect(s) of particular herbs. However, when used in new combinations, the effects and side-effects of the combination are unpredictable. Accordingly, prior to clinical trialling a formulation comprising a combination of at least Radix rehmanniae praeparata, Rhizoma ligustici chuanxiong, Radix angelicae sinensis, Folium artemisiae argyi, Ramulus cinnamomi, Poria cocos and Radix paeoniae alba, it was not known which combination of herbs would result in a formulation that most effectively reduced menstrual-related symptoms.

### Example 2

### Manufacturing of formulation comprising extract of combination of herbs

### Introduction

Traditional methods of preparing Chinese medicines typically involve grinding dried herbs into a powder, which are rolled into small, black round pills; bound together with honey; or alternatively, by decocting dried herbs in boiling water once or twice, typically for between 30 min and 1 hour. It was found that traditional methods of preparing and/or decocting the herbs for the present formulation did not provide a formulation with consistent potency. Additionally, there were reservations regarding the presentation of the medications (eg decoction or rolled pills) amongst the subjects. Accordingly, the present inventor designed an alternative preparation method.

### Materials and Methods

The dried herbs were obtained from a reliable, reputable source from He bei, China. Each of the herbs sourced were of high quality as shown by quality and microbiological assays performed by the supplier. Assays generally include checking for suitable physical characteristics (colour, appearance, odour), absence of heavy metals (arsenic, lead and mercury), and absence of microbial contamination (eg moulds, Salmonella, Escherichia coli, Staphylococcus aureus, Pseudomonas aeroginosa, and/or Asperigillus sp.) as appropriate. The shelf-life of such herbs is two years when stored in tightly closed containers free of excess heat, moisture, light or air, in a cool, dry place, away from direct sunlight.

An extract of a combination of herbs was prepared by mixing dried, ground herbs together in predetermined weight/weight ratios prior to extraction. The dried herb combination was then immersed in 75% ethanol for 3 hours at 50° C. in tanks containing pieces of unglazed earthenware under vacuum conditions (−50 cm Hg) using a Joharprima Macerator-Extractor. The mixture was then macerated for one week. It is thought that the use of earthenware in the process of maceration facilitates enhanced absorption of the herbal contents in the stomach. The mixture was machine pressed and filtered (Joharprima Extractor) The resulting liquid extract (miscella) was concentrated by evaporation at 55° C. under vacuum conditions (−50 cm Hg). The concentrate was then dried in a drying oven at 50° C. The dried extract was then milled to fine granules of less than 1.0 mm in size.

Dicalcium phosphate dibasic (DCPD) filler and excipients (sodium starch glycolate, magnesium stearate and fumed silica absorbent) were mixed in a standard ratio. The filler/excipient mixture (19.5 mg) was added to 630.5 mg of the herbal extract to produce 650 mg capsules. No, preservatives or artificial colourings were added. The same ratio of filler/excipient to herbal extract was used to make capsules of 500 or 250 mg (ie 3% filler/excipient).

### Results and Discussion

The specific method of preparation and extraction of the herbal formulation advantageously provides consistent distribution of the extracts of the various herbs in the formulation, such that each capsule advantageously provides consistent potency and, accordingly, effective dosages. Capsules provide the formulation in a form that is considered to be a more acceptable oral form than, for example, rolled pills.

### Example 3

### Clinical Trial of Formulation Comprising Extract of Combination of Herbs

### Materials and Methods

Clinical Trial Methodology—

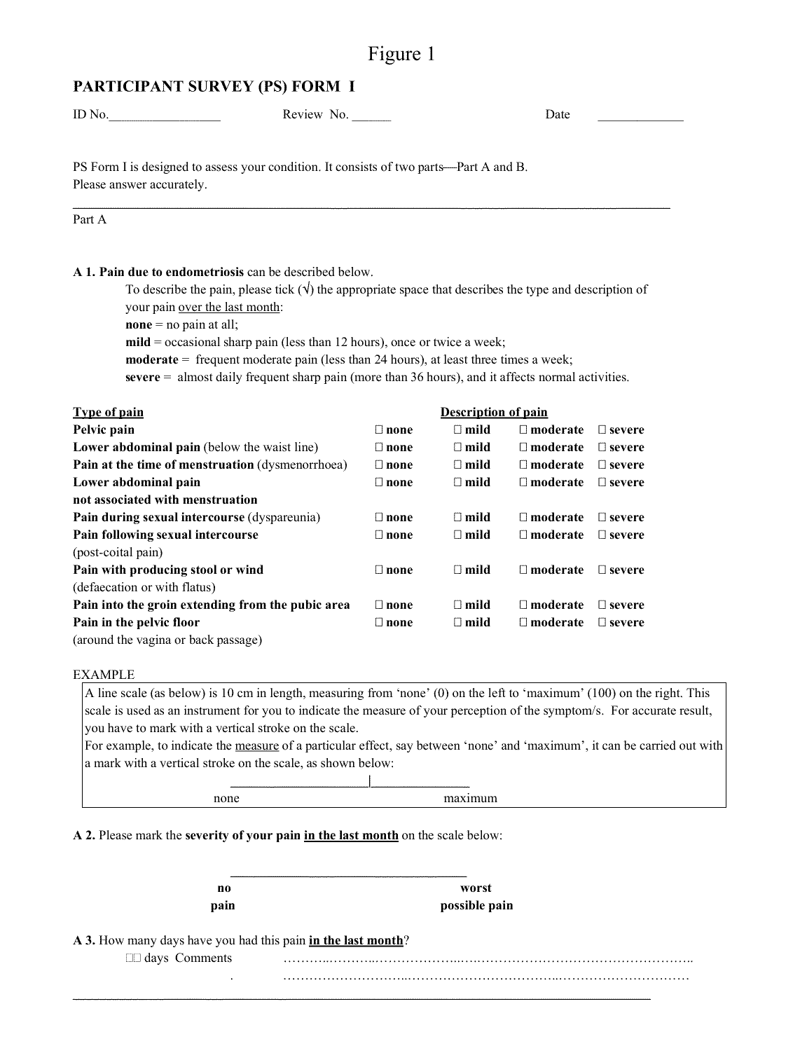

A randomised, placebo-controlled, double-blind, multicentre clinical trial was carried out. The inclusion criteria were women (18-44 years) with laparoscopy-proven endometriosis and moderate to severe menstrual pain of more than six months in duration and a visual analogue scale (VAS) pain score of greater than 30 (see FIG. 1, A2 for an example of VAS assessment). The exclusion criteria were women with diabetes mellitus, malignancies, immune deficiencies or treatments, and anti-depressant medications. Outcomes measured were pain intensity (measured with VAS), restriction of activities (using the Likert scale) and safety of treatment (ie assessment for side-effects), incorporated in a self-reported Participant Survey (PS) Form I (FIG. 1). Safety of treatment was additionally monitored at each follow-up assessment by clinical observations for signs and symptoms indicative of drug sensitivities or reactions including gastrointestinal symptoms (eg nausea, vomiting, and/or changes to bowel motions), gynaecological symptoms (eg vaginal dryness, changes to menstrual flow and/or duration), and/or specific symptoms (eg rash, dizziness and/or flushing). Of 125 eligible women, 100 were recruited and randomised to either a herbal formulation or a placebo treatment group (50 women in each group). This sample size permitted a power for a treatment effect of 30% (α=0.05; β=0.10), allowing for withdrawals. Intention-to-treat analysis was used. Randoinisation was computer-generated by a neutral person not involved in the trial, and the allocation sequence of the treatment groups was maintained until completion of the analysis. Blinding of the researchers and participants to the allocation of the treatment groups, together with, masking of the interventions labelled with serial identification numbers, ensured double-blinding. The women were assessed monthly for the six-month treatment period and at a follow-up after a two-month medication-free period. Compliance of the treatment was assessed with pill count and a self-reported log card provided at each assessment.

Formulation—

The herbal formulation tested was prepared in capsule form as described in Example 2 using the combination and proportions of herbs as shown in Table 1. Each capsule had a weight of 500 mg.

TABLE 1
Composition of herbal formulation

| Plant/herb | Chinese/Pinyin name | % weight* |
|---|---|---|
| Radix rehmanniae praeparata | Shu di | 12 |
| Rhizoma ligustici chuanxiong | Chuan xiong | 10 |
| Radix angelicae sinensis | Dang gui | 10 |
| Rhizoma atractylodis macrocephalae | Bai zhu | 10 |
| Ramulus cinnamomi | Gui zhi | 10 |
| Poria cocos | Fu ling | 10 |
| Radix paeoniae alba | Bai shao | 8 |
| Cortex moutan radicis | Mu dan pi | 8 |
| Radix paeoniae rubra | Chi shao | 8 |
| Folium artemisiae argyi | Ai ye | 4 |
| Radix et rhizoma rhei | Da huang | 4 |
| Radix glycyrrhizae praeparata | Zhi gan cao | 6 |

*w/w of total combined dried herbs prior to extraction

Placebo—

The placebo consisted of pre-gelatinised cornflour with added trace amounts of plant pigments (anthocyanins, carotenoids and chlorophyll) resulting in a dark brown colouring. No preservatives or artificial colouring were added. It was packed into capsules identical in size and colour to the herbal intervention.

Dosage—

Each woman in the trial took 10×500 mg capsules per day for six months.

### Results

Study Compliance and Completion—

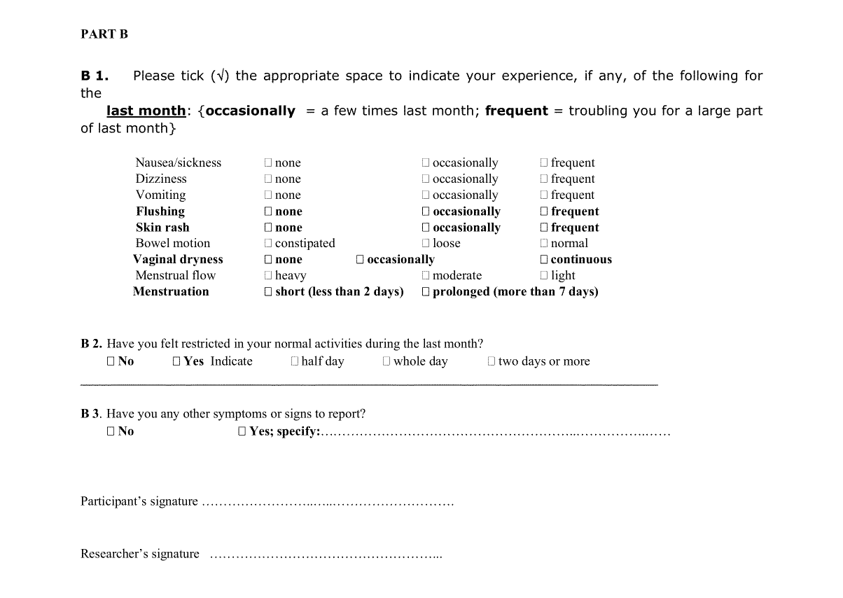

Of 100 women, 76 completed the eight-month study, 19 withdrew and 5 became pregnant (see FIG. 2). The number of pregnancies in each group was comparable, with three women becoming pregnant in the herbal formulation group and two in the placebo group. The allocation sequence (ensuring double-blinding) was maintained until completion of the analysis. Compliance of the treatments in the women was statistically satisfactory.

Effectiveness—

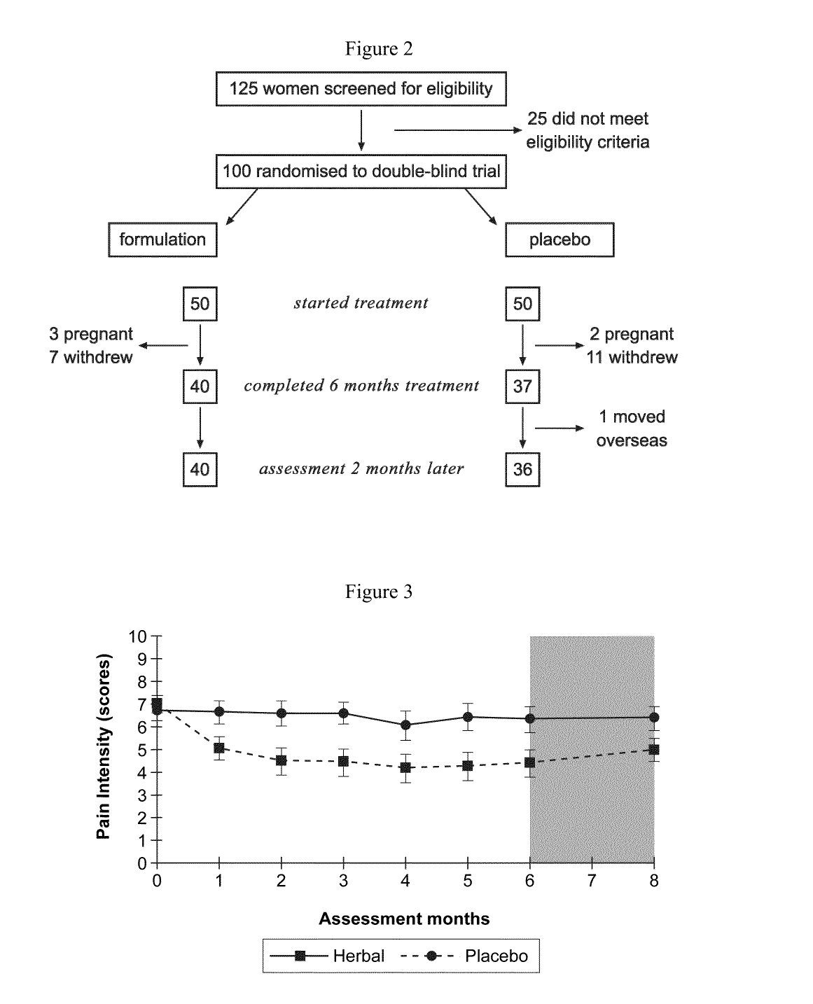

The herbal formulation group demonstrated reduced mean pain intensity compared with the placebo group (p<0.05; Table 2 and FIG. 3). Linear regression of the pain scores over the six months of the trial shows that the herbal formulation group had significantly reduced mean pain scores compared to the placebo group (p<0.001), with a partial correlation of 0.50 (95% CI 0.39-0.58; Table 3), meaning that the herbal formulation intervention demonstrated a medium effect size with regard to pain relief.

TABLE 2
Pain scores between groups over time

| Assessment time (months) | | | | | | | | |
|---|---|---|---|---|---|---|---|---|
| Baseline | 1 | 2 | 3 | 4 | 5 | 6 | 8^{b} | Trend test^{a} |

Placebo group (n = 50)^{†}

| | | | | | | | | | |
|---|---|---|---|---|---|---|---|---|---|
| Mean | 6.75 | 6.66 | 6.61 | 6.62 | 6.07 | 6.44 | 6.35 | 6.42 | Pearson's |
| SD | 1.51 | 1.70 | 1.90 | 1.76 | 2.15 | 2.13 | 2.12 | 1.92 | r = −0.08 |
| 95% CI | (6.3, 7.2) | (6.2, 7.2) | (6.1, 7.2) | (6.1, 7,1) | (5.4, 6.7) | (5.8, 7.0) | (5.8, 6.9) | (5.9, 7.0) | |
| p value ^{c} | — | NS | NS | NS | * | NS | NS | NS | p-value |
| | | | | | | | | | for r: NS |

Herbal group (n = 50)^{†}

| | | | | | | | | | |
|---|---|---|---|---|---|---|---|---|---|
| Mean | 7.03 | 5.09 | 4.50 | 4.45 | 4.20 | 4.28 | 4.39 | 4.99 | Pearson's |
| SD | 1.31 | 1.79 | 1.90 | 1.92 | 2.00 | 2.12 | 2.15 | 2.27 | r = −0.37 |
| 95% CI | (6.7, 7.4) | (4.6, 5.6) | (3.9, 5.1) | (3.9, 5.0) | (3.6, 4.8) | (3.6, 4.9) | (3.8, 5.0) | (4.5, 5.5) | |
| p value ^{d} | — | *** | *** | *** | *** | *** | *** | *** | p-value |
| | | | | | | | | | for r: *** |
| p-value ^{e} | NS | *** | *** | *** | *** | *** | *** | *** | |

^{a}Pearson's r computed by correlating individual's pain score of the assessment month from baseline, up to 6 months
^{b}After a two-month medication-free period
^{c} p value for paired t-tests between baseline and assessment month
^{d} p value for paired t-tests between baseline and assessment month
^{e} p value for independent t-tests between groups
* Significant (p < 0.05);
*** Significant (p < 0.001) (Pearson's correlation r = 0.37)
NS Not significant (p > 0.05)
^{†}Note:
Observations carried out for 6 months (i.e. baseline plus 6 months of medication period: 350 observations in each group based on ITT analysis)

TABLE 3
Pain score^{a} over six months of intervention between
treatment groups by linear regression analysis

| Independent | Unstandardised | Standardised | | Partial |
|---|---|---|---|---|
| variables | coefficients | coefficients | p value | correlation |
| Month of | 0.23 | 0.23 | <0.001 | |
| intervention | | | | |
| Herbal vs. | 1.98 | 0.48 | <0.001 | 0.50 (95% CI |
| placebo | | | | 0.39~0.58) |

^{a}Pain score was determined by subtracting individual pain score at assessment month from baseline

The reduced pain intensity observed in the herbal formulation group correlated with reduced days with pain (odds ratio r=0.37; p<0.001; Table 4). Specifically, the herbal formulation group demonstrated a moderate correlation between reduced number of days with pain per month and mean pain scores (Pearson's correlation r=0.37), compared to placebo (r=0.20).

TABLE 4
Correlation between number of days with pain
per month and pain intensity over 8 months

| Treatment | Number of | Pearson's | |
|---|---|---|---|
| group | observations | correlation (r) | p value |
| Placebo | 400 | 0.20 | <0.001 |
| Herbal | 400 | 0.37 | <0.001 |

Women in the herbal formulation group reported less restriction of activities and reduced number of days with restriction compared to the placebo group (odds ratio r=3.30; p<0.001; Table 5).

TABLE 5
Odds ratio of reduction in restriction of activities^{a} during six
month trial between groups by logistic regression analysis

| Independent variables | Odds ratio (Herbal) | p value |
|---|---|---|
| Month of intervention | 1.17 | <0.001 |
| Herbal vs. Placebo | 3.30 | <0.001 |

^{a}Reduced restriction of activities from baseline

Table 5 shows that the odds ratio estimate of reduced restriction of activities over the six months of the trial in the herbal formulation group compared to the placebo group was 3.30 (p<0.001). Sustained treatment effect (measured by reduced pain intensity) was demonstrated throughout the six-month trial and at eight months, ie two months after completion of treatment (Pearson's correlation τ=−0.37; p<0.001; see Table 2 and FIG. 3), when participants were assessed for effectiveness and safety of the intervention (see PS Form I, FIG. 1). This sustained effect after a two-month treatment-free period demonstrated that the herbal formulation was effective at reducing pain associated with endometriosis and safe for prolonged usage with no significant side-effects. Further, these results establish that the herbal formulation could treat the underlying causation of menstrual-related pain and symptoms. No clinical side effects or abnormalities were detected as determined by the safety profile of symptoms (p>0.001; Tables 6 and 7), clinical observations and regular blood tests.

TABLE 6
Decrease in frequency^{a} of general symptoms over six months of
intervention between groups by linear regression analysis

| | Herbal formulation vs. placebo | | | |
|---|---|---|---|---|
| | Unstandardised | Standardised | | Partial |
| Symptoms | coefficients | coefficients | p value | correlation |
| Nausea | 0.07 | 0.05 | NS | 0.01 |
| Dizziness | 0.04 | 0.03 | NS | 0.03 |
| Vomiting | −0.003 | −0.004 | NS | −0.004 |
| Flushing | 0.01 | 0.01 | NS | 0.01 |
| Skin rashes | 0.03 | 0.04 | NS | 0.04 |
| Bowel motion | 0.09 | 0.10 | NS | 0.10 |

^{a}Decrease in frequency computed by subtracting individual's frequency at assessment month from baseline
NS Not significant (p > 0.05)

TABLE 7
Decrease in frequency^{a} of menstrual or gynaecological
symptoms over six months of intervention between
groups by linear regression analysis

| | Herbal vs. placebo | | | |
|---|---|---|---|---|
| | Unstandardised | Standardised | | Partial |
| Symptoms | coefficients | coefficients | p value | correlation |
| Vaginal dryness | −0.08 | −0.09 | NS | −0.09 |
| Menstrual flow | −0.11 | −0.10 | NS | −0.10 |
| Menstruation days | 0.07 | 0.06 | NS | 0.06 |

^{a}Decrease in frequency computed by subtracting individual's frequency at assessment month from baseline
NS Not significant (p > 0.05)

Natural Fertility—

The women's natural fertility was not compromised by the trial as there was no significance difference in the number of pregnancies between the groups. Five women became pregnant during the six-month trial, three of whom were in the herbal formulation group, and two of whom were in the placebo group (see FIG. 2).

Generalisation of Clinical and Statistical Findings—

The rigorous setup and conduct of the study demonstrated practicality and indicate that the clinical and statistical findings can be generalised (Pringle and Churchill, 1995) to women of reproductive age, in healthcare settings ranging from primary healthcare settings (eg first level care provided by health services and systems including community clinics and services, backed up by general practice, medical doctors and trained medical staff) up to tertiary healthcare settings (eg specialised, highly technical level of health care that includes diagnosis and treatment of disease and disabilities, including hospitals, intensive care units, advanced diagnostic support services, and specialised personnel etc) (Roberts et al., 2001; Helms, 2002). Other menstrual conditions share similar symptoms with endometriosis. The inclusion-exclusion criteria of the trial included menstrual-related pain and symptoms, which also occur in other benign menstrual disorders such as dysmenorrhoea, irregular menstrual bleeding including amenorrhoea and menorrhagia, and dyspareunia. This trial is therefore indicative of treatment of these conditions. The eight-month study duration and robust compliance indicated safe and acceptable long-term usage of the formulation for treating menstrual-related conditions.

Generalisation of the clinical and statistical findings was substantiated by the statistical target population with comparable conditions and settings. Statistically, the target population was robust because the trial had (a) sufficient power due to the number of recruited participants with suitable criteria for recruitment and analysis; (b) suitable end-points and inclusion-exclusion criteria; (c) sufficient and suitable data collection and analysis due to satisfactory compliance; (d) double-blinding and masking to prevent bias; (e) multiple observations (monthly assessments during the six-month trial and a follow-up assessment two months after the completion of the trial); and (f) placebo as the comparator. The target population was women with specific inclusion-exclusion criteria. The inclusion criteria were women of reproductive age suffering from menstrual-related pain and symptoms for more than six months, and the exclusion criteria were malignancies, immune conditions or treatments, or diabetes mellitus. Comparable conditions of menstrual-related pain and symptoms for six months or more in women of reproductive age occur in other benign gynaecological conditions such as dysmenorrhoea.

### Discussion

The results show that the herbal formulation provides an effective and safe means for treating menstrual-related conditions applicable throughout the oestrous cycle to reduce pain associated with menstrual-related conditions by addressing the underlying cause of the menstrual-related conditions according to Traditional. Chinese Medicine (TCM) principles. The findings of the trial provide evidence of the effectiveness and safety of the formulation of the invention in treating menstrual-related pain and symptoms, generalised to women of reproductive age in the wider population, including other benign menstrual-related pain conditions such as dysmenorrhoea and menstrual irregularities.

For practical purposes, the herbal formulation is suitable for distribution in primary to tertiary healthcare settings because it can be orally administered. Moreover, the present inventor has realised that the formulation advantageously derives synergistic action as a result of the manufacturing process.

### Example 4

### Alternative Method of Manufacturing Formulation Comprising Extract of Combination of Herbs

Herbs were sourced as described in Example 2. An extract of a combination of herbs was prepared by mixing dried herbs together in predetermined weight/weight ratios as shown in Table 1 prior to extraction. The dried herb whole plant or parts were sliced and/or smashed to produce small coarse pieces of approximately 2-3 cm in length. The coarse herb pieces were immersed in 75% ethanol for 30 minutes at 50° C. under standard vacuum conditions. The extraction, process was then continued for 2 hours at 50° C. without vacuum. The mixture was then macerated in tanks containing earthenware for 36 hours. It is thought that the use of earthenware in the process of maceration facilitates enhanced absorption of the herbal contents in the stomach. The liquid extract was then filtered, and the filtered extract was dried by evaporation at 50° C. for 3 hours under standard vacuum conditions. The resulting dried product was ground to fine granules of less than 1.0 mm size.

Dicalcium phosphate dibasic (DCPD) filler and excipients (sodium starch glycolate, magnesium stearate and fumed silica absorbent), combined as described in Example 2, were added at a ratio of 19.5 mg per 630.5 mg herbal extract to produce 650 mg capsules. The same ratio can be used to produce capsules of other sizes. No preservatives or artificial colourings were added.

### Example 5

### Manufacturing Formulation Comprising Combination of Herbs as Rolled Pills

Dried herbs were sourced as described in Example 2 and combined in the ratio described in Table 1. Rolled pills were manufactured using traditional methods known to those skilled in the art.

### Example 6

### Manufacturing Formulation Comprising Combination of Herbs as Decoction

Dried herbs were sourced as described in Example 2 and combined in the ratio described in Table 1. The herbs were decocted using traditional methods known to those skilled in the art.

### Prophetic Example 1

### Formulation Containing a Combination of Seven, Primary Herbs

The formulation can be prepared in capsule form as described in Example 2 or 4, using the combination and proportions of herbs as shown in Table 8.

TABLE 8
Composition of herbal formulation

| Plant/herb | Chinese/Pinyin name | % weight* |
|---|---|---|
| Radix rehmanniae praeparata | Shu di | 18-21% |
| Rhizoma ligustici chuanxiong | Chuan xiong | 13-16% |
| Radix angelicae sinensis | Dang gui | 13-16% |
| Folium artemisiae argyi | Ai ye | 6-7% |
| Ramulus cinnamomi | Gui zhi | 15-16% |
| Poria cocos | Fu ling | 15-16% |
| Radix paeoniae alba | Bai shao | 12-14% |

*approximate w/w of total combined herbs prior to extraction

This formulation can be used to treat women with menstrual-related conditions in a similar manner as described in Example 3.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

1. Bensky, D. & Gamble, A. (1986) Chinese Herbal Medicine Materia Medica. Eastland Press, Seattle.
2. Chi, C. (1994). Social Science & Medicine, 39, 307.
3. Fu, Q. Z., trans. Yang, S. & Liu, D. (1995) Fu qing-zhu's gynaecology, Blue Poppy Press, Boulder, Mich.
4. Gottlieb, S. (2000). British Medical Journal, 320, 1623.
5. He, H., et al. (1994) Journal of Nanjing College of Traditional Chinese Medicine (Nan Jing Zhong Yi Xue Yuan Xue Bao), 10, 16-17,
6. He, H., Li, S. & Border, C. (2006) Cochrane Database of Systematic Reviews, 4.
7. Helms P. J. (2002) Pediatr Allergy Immunol, 13, 4-9.
8. Hsu, H. Y. (1986) Oriental materia medica, a concise guide. Long Beach, Calif., Oriental Healing Arts Institute, 144-5.
9. Jiang, J., et al. (1998) Chin Folk Ther [Zhong guo min jian liao fa], 5, 35-36.
10. Kotani, N., et al. (1997) Am J Chin Med, 25(2), 205-12.
11. Liu, M. R. & Wu, K. M. (1999) Infertility. In: Zhang, Q. W. editor(s). Gynaecology in World Traditional Medicine. 1st Edition. Beijing: Science Press, 4189.
12. Mu, G. (2000) Tradit Chin Med in Hansu (Han Su Zhong Yi), 4, 47-48.
13. Ou, M. (1992) Chinese-English Manual of Common-used in Traditional Chinese Medicine. Guangzhou: Guangdong Science and Technology Press.
14. Page, R. L. & Lawrence, J. D. (1999) Pharmacotherapy, 19, 870-6.
15. Pringle, M. & Churchill, R. (1995) BMJ, 311, 1382-3
16. Roberts, M. S., et al. (2001) Br J Clin Pharmacol, 51, 257-65.
17. Schwartz, C. (1994) Chinese Herbology in Veterinary Medicine. In: Veterinary Acupuncture: Ancient Art to Modern Medicine pp. 691-697 (Edited by Schoen, A. M., American Veterinary Publications Inc., 5782 Thomwood Dr., Goletta, Calif. 93117).
18. Shi, D., et al. (2000) Chin Herb Mede (Zhong Cao Yao), 31(5), 365.
19. St, Y. & Yang, J. L. (2000) Infertility: In Traditional Chinese Medicine for Gynaecological Disease (Eds, Li, L. Y. & Xu, M.). People's Health, Beijing, 1, 410-1.
20. Tomlinson, B., et al. (2000) Toxicity of complementary therapies: an eastern perspective. J Clin Pharmacol, 40, 451-6.

1. A formulation comprising a combination of herbs or extract(s) thereof for treating a menstrual-related condition, the combination comprising Radix rehmanniae praeparata, Rhizoma ligustici chuanxiong, Radix angelicae sinensis, Folium artemisiae argyi, Ramulus cinnamomi, Poria cocos and Radix paeoniae alba; optionally in combination with a pharmaceutically-acceptable carrier or excipient.

2. The formulation of claim 1, the combination further comprising at least one herb or extract thereof selected from the group consisting of Cortex moutan radicis, Radix paeoniae rubra, Rhizoma atractylodis macrocephalae, Radix et rhizoma rhei, Radix bupleuri, Rhizoma cyperi, and Radix glycyrrhizae praeparata.

3. The formulation of claim 1, the combination further comprising Cortex moutan radicis, Radix paeoniae rubra, Rhizoma atractylodis macrocephalae, Radix et rhizoma rhei, Radix bupleuri, Rhizoma cyperi and Radix glycyrrhizae praeparata.

4. The formulation of claim 1, the combination further comprising Cortex moutan radicis, Radix paeoniae rubra, Rhizoma atractylodis macrocephalae, Radix et rhizoma rhei and Radix glycyrrhizae praeparata.

5. The formulation of claim 1, wherein the combination comprises approximately 2% to 30% w/w Radix rehmanniae praeparata, approximately 0.1% to 25% w/w Rhizoma ligustici chuanxiong, approximately 0.1% to 25% w/w Radix angelicae sinensis, approximately 0.1% to 20% w/w Folium artemisiae argyi, approximately 0.1% to 25% w/w Ramulus cinnamomi, approximately 0.1% to 25% w/w Poria cocos, approximately 0.1% to 25% w/w Radix paeoniae alba, approximately 0% to 20% w/w Cortex moutan radicis, approximately 0% to 20% w/w Radix paeoniae rubra, approximately 0% to 20% w/w Rhizoma atractylodis macrocephalae, approximately 0% to 15% w/w Radix et rhizoma rhei, approximately 0% to 15% w/w Radix bupleuri, approximately 0% to 15% w/w Rhizoma cyperi and approximately 0% to 15% w/w Radix glycyrrhizae praeparata.

6. The formulation of claim 1, wherein the combination comprises 8% to 31% w/w Radix rehmanniae praeparata, 3% to 26% w/w Rhizoma ligustici chuanxiong, 3% to 26% w/w Radix angelicae sinensis, 0.1% to 17% w/w Folium artemisiae argyi, 5% to 26% w/w Ramulus cinnamomi, 5% to 26% w/w Poria cocos and 2% to 14% w/w Radix paeoniae alba.

7. The formulation of claim 3, wherein the combination comprises 2% to 22% w/w Radix rehmanniae praeparata, 0.1% to 20% w/w Rhizoma ligustici chuanxiong, 0.1% to 20% w/w Radix angelicae sinensis, 0.1% to 14% w/w Folium artemisiae argyi, 0.1% to 20% w/w Ramulus cinnamomi, 0.1% to 20% w/w Poria cocos, 0.1% to 18% w/w Radix paeoniae alba, 0.1% to 18% w/w Cortex moutan radicis, 0.1% to 18% w/w Radix paeoniae rubra, 0.1% to 20% w/w Rhizoma atractylodis macrocephalae, 0.1% to 14% w/w Radix et rhizoma rhei and 0.1% to 16% w/w Radix glycyrrhizae praeparata.

8. The formulation of claim 4, wherein the combination comprises 2% to 22% w/w Radix rehmanniae praeparata, 0.1% to 18% w/w Rhizoma ligustici chuanxiong, 0.1% to 18% w/w Radix angelicae sinensis, 0.1% to 14% w/w Folium artemisiae argyi, 0.1% to 19% w/w Ramulus cinnamomi, 0.1% to 19% w/w Poria cocos, 0.1% to 18% w/w Radix paeoniae alba, 0.1% to 18% w/w Cortex moutan radicis, 0.1% to 18% w/w Radix paeoniae rubra, 0.1% to 18% w/w Rhizoma atractylodis macrocephalae, 0.1% to 14% w/w Radix et rhizoma rhei, 0.1% to 14% w/w Radix bupleuri, 0.1% to 14% w/w Rhizoma cyperi and 0.1% to 16% w/w Radix glycyrrhizae praeparata.

9. The formulation of claim 1, wherein the formulation comprises extract(s) derived from the combination of herbs.

10. The formulation of claim 1, wherein the formulation comprises an extract derived from the combination of herbs, wherein the combination of herbs are mixed prior to extraction and extracted together.

11. The formulation of claim 9, wherein the extract(s) is prepared by immersing the combination of herb(s) in an alcohol, macerating the immersed herb(s) to obtain a herbal alcohol solution, and then drying the herbal alcohol solution to obtain the extract(s).

12. The formulation of claim 1, wherein the formulation is provided in capsule or tablet form, or is within a sachet.

13. A method of treating a menstrual-related condition comprising administering to a female subject suffering from the menstrual-related condition an effective amount of the formulation of claim 1.

14. The method of claim 13, wherein the formulation is administered at a dosage within the range of 1850 mg to 7050 mg of active components per subject per day, wherein the active components consist of the combination of herbs or extract(s) thereof.

15. The method of claim 14, wherein the formulation is administered at a dosage within the range of 3150 mg to 5050 mg of active components per subject per day.

16. The method of claim 15, wherein the method reduces pain associated with the menstrual-related condition.

17. The method of claim 16, wherein the menstrual-related condition is selected from the group consisting of endometriosis, dysmenorrhoea, irregular menstrual bleeding, and dyspareunia.

18. The method of claim 14, wherein the formulation is administered throughout the oestrous cycle.

19. The method of claim 14, wherein the formulation is administered orally.
